(19) United States
(12) Patent Application Publication
(10) Pub. No.: US 2012/0271259 A1
(43) Pub. Date: Oct. 25, 2012

(54) Method and Apparatus for Eliminating Urine and Keeping a Person Dry

(76) Inventor: Izaak Alan Ulert, Houston, TX (US)

(21) Appl. No.: 13/223,304

(22) Filed: Aug. 31, 2011

Related U.S. Application Data
(60) Provisional application No. 61/477,892, filed on Apr. 21, 2011.

(51) Int. Cl. A61M 1/00

(52) U.S. Cl. 604/327

(57) ABSTRACT

An apparatus for eliminating excreted urine so as to keep a person dry without the person having to actively or consciously participate in the process. The apparatus may be part of a diaper or similar article of clothing or may be a device used in such clothing. The apparatus has a plurality of receiving tubes for receiving the urine, a central collecting tube and/or similar collecting and holding facility associated with the receiving tubes for receiving, collecting and at least temporarily holding the urine, and a vacuum source for suctioning the urine into the receiving tubes. A sensor detects urine released by the person and activates the vacuum source which pulls the urine into the receiving tubes. The central collecting tube may be routed directly or indirectly to a drain for disposing of the collected urine.

### RELATED APPLICATION

The present application claims priority from U.S. Patent Provisional Patent Application Ser. No. 61/477,892, filed Apr. 21, 2011.

### FIELD OF THE INVENTION

The present invention relates to methods and apparatus for collecting and eliminating urine from a person. More particularly, the present invention relates to disposable articles having a responsive system that includes an actuator having utility in collecting and eliminating urine upon excretion by a human being.

### BACKGROUND OF THE INVENTION

Today, disposable articles such as diapers and adult incontinence briefs are widely used in the care of hospital patients, immobile patients, and incontinent adults as a means of containing, isolating and disposing of bodily wastes. These articles are generally preferred over catheters and penis caps for collecting urine although the latter still have usage for certain applications, particularly in hospitals.

While many advancements have been made in the field of disposable articles for both infants and adults, which have enabled them to become widely preferred over conventional cloth garments, a number of problems still exist. Among the problems experienced with disposable diapers are leakage of bodily waste (e.g., urine, feces, menses), skin rash and irritation, difficult cleanup, waste odor, and lack of fit customization to individuals.

Various attempts have been made to address these problems. Super absorbent polymers, for example, have been used to increase the ability of an absorbent article to absorb and retain urine. Barrier leg cuffs have also been used to improve fit and reduce leakage. U.S. Pat. No. 3,860,003, entitled “Contractible Side Portions For Disposable Diaper,” issued to Kenneth B. Buell on Jan. 14, 1975, for example, describes an elasticized leg cuff disposable diaper that has achieved wide acceptance and commercial success. In addition, self-contracting leg gathers have been disclosed that react with a liquid activator such as water or urine. See for example, U.S. Pat. No. 4,246,900, entitled “Diaper Including Moisture-Responsive Seal Means,” issued to Friedrich-Wilhelm Schroder on Jan. 27, 1981. These self-contracting gathers have the problem that they either require action by the caregiver to activate the components or operate continuously and require too much of a liquid activator to fully contract because the contraction is proportional to the amount of the liquid activator.

There continues to be a need for improved diapers, adult incontinence briefs and other means for collecting and eliminating urine and keeping a person dry.

### SUMMARY OF THE INVENTION

The apparatus of the present invention eliminates urine for keeping a person dry without the person having to actively or consciously participate in the process. The apparatus may comprise part of a diaper or similar article of clothing or may be a device used in such clothing. The apparatus comprises a plurality of receiving tubes for receiving the urine, a central collecting tube and/or similar collecting and holding facility associated with the receiving tubes for receiving, collecting and at least temporarily holding the urine, and a vacuum source for suctioning the urine into the receiving tubes.

The urine may be routed directly via the central collecting tube, or indirectly as through a holding container, from the receiving tubes to a drain. A sensor detects urine released by a person and activates a vacuum source which pulls the urine into the receiving tubes. The central collecting tube may be routed directly or indirectly to a drain for disposing of the collected urine. Preferably, the vacuum source is associated with a heater or other means for warming air associated with the vacuum. Warm air is more comfortable to the person than cold air, provided it is not so hot as to burn the person. Thus, the heater must not heat the air to temperatures that would burn or be uncomfortable.

### BRIEF DESCRIPTION OF THE DRAWINGS

FIG. 1 is a plan view of an article of the present invention, which is shown a diaper embodiment, in a flat-out state with portions of the structure being cut-away to more clearly show the construction of the article.

FIG. 2 is a side view of an enlarged portion of FIG. 1 showing a plurality of receiving tubes feeding into a central receiving or container tube.

FIG. 3 is a frontal side view of a plurality of receiving tubes feeding into a central receiving or container tube, in an alternative configuration to that shown in FIG. 2, for an article of the invention.

### DESCRIPTION OF PREFERRED EMBODIMENTS OF THE INVENTION

The present invention is directed to an article having a responsive system that includes a sensor and actuator with an associated vacuum (preferably with warm air) that acts in response to an input from the presence of moisture from urine (or a component of the urine) for activation. The article of the invention is a device which is placed against or in proximity to the body of the wearer and which uses vacuum to collect the urine discharged from the body into a plurality of tubes which in turn direct the urine into a container tube for disposal into a collection container or to a drain.

The term “disposable” is used herein to describe articles which generally are not intended to be laundered or otherwise restored or reused (i.e., they are intended to be discarded after use and, preferably, to be recycled, composted or otherwise disposed of in an environmentally compatible manner). As used herein, the term “disposed” is used to mean that an element(s) of the article is formed (joined and positioned) in a particular place or position as a unitary structure with other elements of the article or as a separate element joined to another element of the article. As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element. A “unitary” article refers to articles which are formed of separate parts united together to form a coordinated entity so that they do not require separate manipulative parts like a separate holder and liner.

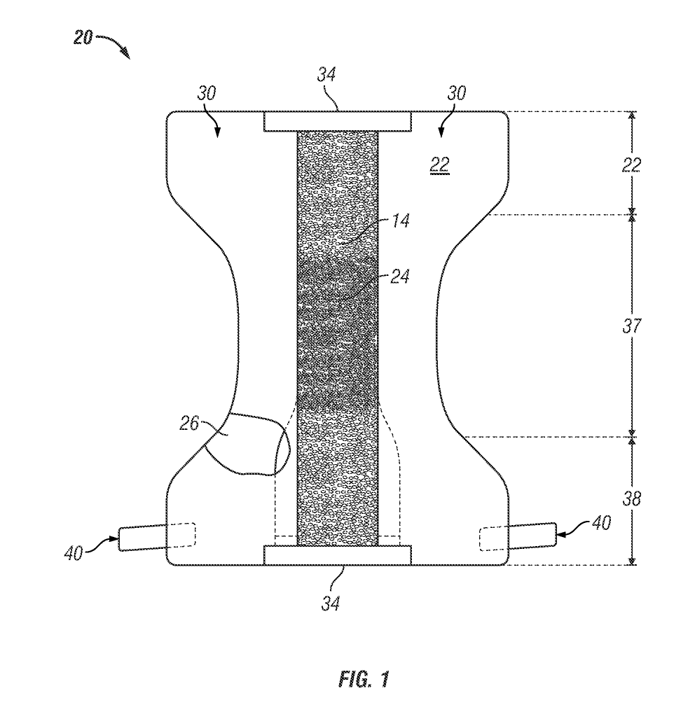

A preferred embodiment of an article of the present invention is a unitary, preferably disposable article, article 20, shown in FIG. 1, like a “diaper” generally worn by infants and incontinent persons about the lower torso. The present invention is also applicable to other absorbent and non-absorbent articles such as incontinence briefs, incontinence undergarments, absorbent inserts, diaper holders and liners, feminine hygiene garments, tampons, wipes, disposable towels, tissues, water absorbing articles, oil absorbing articles, spill cleanup bags, disposable mops, and the like.

FIG. 1 is a plan view of an article 20 of the present invention, which is shown in this Figure as a diaper, in a flat-out, state with portions of the structure being cut-away to more clearly show the construction of the article 20. This diaper configuration is not intended to be limiting and it is contemplated that the article of the invention could be a self-contained portable device for positioning in traditional undergarments.

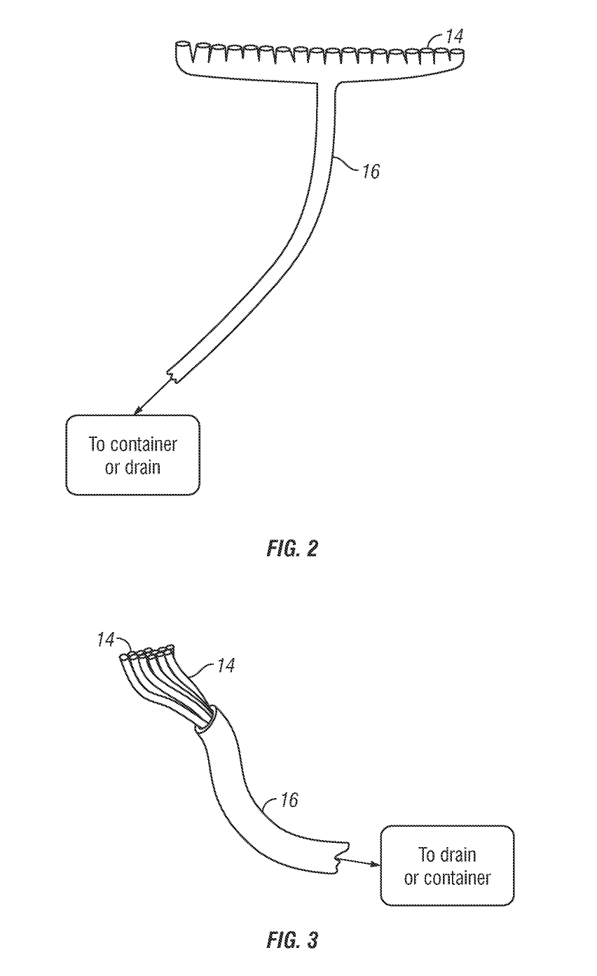

The portion of the article 20 which faces the wearer is oriented towards the viewer. As shown in FIG. 1, the article 20 preferably comprises a liquid pervious topsheet 24 which comprises a plurality of receiving tubes 14 (shown in FIG. 2 and in an alternative configuration in FIG. 3) which feed into a central receiving or container tube 16; a vacuum or suction source (not shown) for injecting air into the plurality of tubes 14 and pulling urine contacting one or more of the plurality of tubes 14 into said tubes 14; a power source (not shown) for the vacuum source; a sensor (not shown) for activating the vacuum source or for activating an activator (not shown) for activating the vacuum source; and preferably a heat source (not shown) for warming the air injected into the plurality of tubes 14 preferably to body temperature or a temperature that would be comfortable to a human being and would not cause burns. The article 20 may also optionally comprise a liquid impervious backsheet 26 to support the central receiving tube 16; optional side panels 30; optional elasticized leg cuffs (not shown); optional elastic waist feature 34; and a fastening system generally designated 40.

Alternatively, as noted above, the article of the invention may be a portable device that fits into any commercially available traditional disposable diaper or underwear type brief. Such portable device would preferably have means for affixing the device to such diaper or brief, with adhesive on sticky adhesion strips for one of numerous examples. Such portable device (one example depicted in FIG. 3) would preferably be reusable and would comprise a plurality of receiving tubes feeding into a larger receiving or container tube, as with the unitary disposable embodiment of the invention. The larger central tube in turn would feed into a container or into a drain. This embodiment of the invention would also comprise or be associated with a vacuum or suction source for pulling urine into the tubes and a sensor for detecting the urine in proximity to the tubes. The sensor would activate a power source to activate the vacuum or suction source, just as in the first embodiment of the invention. Also preferably, as with the first embodiment of the invention, the device is associated with a source for heating the air. Such heating makes use of the device more comfortable for the human user and is less likely to awaken the human user during sleep.

The configuration and construction of the liquid pervious topsheet 24 which comprises a plurality of receiving tubes 14 may be varied (e.g., the tubes may be evenly or unevenly spaced). However, the total urine withdrawing capacity of the tubes 14 should be compatible with the design loading and the intended use of the article 20.

As used in this application, the term “sensor” refers to a device that is used to detect an event or a parameter that is associated with an event. A parameter associated with an event is any measureable signal that correlates with the occurrence of an event within the frame of reference of the system (i.e., a signal caused by urination). Sensors include anything that responds to one or more specific inputs. Examples of inputs that may be detected by the sensor of the present invention include, but are not limited to, pressure, motion, moisture, enzymes, bacteria, pH, conductivity, resistance, capacitance, inductance, or other chemical, biochemical, biological, mechanical or electrical properties and/or components of bodily wastes, particularly urine. The sensors preferably detect “non-environmental” inputs such as a non-thermal or a non-relative humidity input in order to minimize the number of false responses by minimizing the possibility of an environmental condition triggering the sensor instead of the sensor detecting an input caused by the urine. An electrical or biological sensor may, for example, detect urination by sensing a component of the urine. A sensor may detect one or more events or one or more parameters associated with an event and provide an input to an actuator or a controller. Further, a sensor of the present invention may also be reversible or irreversible. A dissolving film or capsule is an example of an irreversible sensor, while an electrical sensor that detects electrical activity in muscles of the wearer may receive multiple sequential input signals (i.e., is reversible).

As discussed above, sensors of the present invention may include anything that responds to a specific input. For example, the sensor of the present invention may be chemical, mechanical, electrical, etc. A chemical sensor may respond to chemical and/or biochemical inputs such as enzymes typically present in urine, pH, water, biological inputs such as bacteria, or any one or more other components of urine. A chemical sensor may use a chemical reaction as a detection means or may involve a dissolution of a material soluble in an input material of interest. Examples of chemical or biological sensors include dissolving or rupturable films, capsules, cells, seals, etc. that dissolve or rupture in response to a specific chemical, biochemical or biological input or to a specific class of chemical, biochemical or biological inputs. A mechanical sensor may also respond to motion, pressure, etc. An example of a mechanical sensor is a bellows-type in which when a person sits on the sensor the weight pushes down on the bellows to inflate a portion of the sensor. A mechanical sensor may also include a sensor or a portion of the sensor that is broken or separated under a pre-defined applied pressure. An electrical sensor may also be used to respond to moisture, urine, pressure, resistance, capacitance, inductance, etc. An electrical sensor may, for example, include a sensor in which a conductive input from urine completes an electrical circuit; a sensor in which the input such as pressure or tension closes an electrical contact to complete a circuit; a piezoelectric sensor that generates a signal via pressure induced by the wearer or a part of the wearer (e.g., from motion or muscle tone); a sensor in which the resistance, capacitance or inductance varies in the presence of the input to which the sensor responds; or a sensor that receives electrical signals from the body (e.g., from the subcutaneous muscles) of the wearer through a contact such as a skin contact sensor. Optionally, the sensor may be a biosensor as known in the art (e.g., an enzyme sensor, organella sensor, tissue sensor, microorganism sensor, or electrochemical sensor). The sensor may be adapted to detect proteins, sugars, etc. such as described in U.S. Pat. No. 4,636,474 entitled “Toilet Apparatus,” issued to Kenji Ogura et al. on Jan. 13, 1987. Biosensors may comprise bio-recognition systems, typically enzymes or binding proteins such as antibodies immobilized onto the surface of physico-chemical transducers. The biosensors may detect components of urine, such as ammonia and phenol (e.g., via biosensors comprising enzyme electrodes). A specific strain of bacteria may be detected via biosensors employing antibodies raised against that bacterial strain. Exemplary enzyme electrodes that may be used to detect phenols in urine include tyrosinase based electrodes or polyphenol oxidase enzyme electrodes described in U.S. Pat. No. 5,676,820 entitled “Remote Electrochemical Sensor,” issued to Joseph Wang et al. on Oct. 14, 1997 and U.S. Pat. No. 5,091,299 entitled “An Enzyme Electrode For Use In Organic Solvents,” issued to Anthony P. F. Turner et al. on Feb. 25, 1992, respectively.

Optionally, the sensor may be a “proactive sensor” that is capable of detecting changes or signals in or on the body of the wearer, in the article or in the urine, i.e., inputs that directly relate or, at a minimum, correlate to the occurrence of an impending event such as urination or other discharge of bodily waste. A proactive sensor, for example, may detect an impending event such as urination or discharge or a parameter that correlates to such an event. The impending event may be related to the bodily waste, the wearer, the article, or a component or components thereof. A parameter that correlates to an event is any measurable input signal that correlates with the occurrence of the event within the frame of reference of the system (i.e., a signal caused by the urine or the wearer). The proactive sensor may, for example, predict the occurrence of urination or discharge of bodily waste. Proactive sensors in an article may measure many different inputs in order to predict an event. For example, the proactive sensor may monitor a muscle for a relaxation that precedes the release of urine or any other indication that may be used to predict or anticipate the occurrence of urination or a discharge of other bodily wastes.

The sensor may be disposed in and/or operatively connected to any portion of the article of the invention that will be exposed to the input that the sensor is designed to detect. For the purposes of the present invention, the term “operatively connected” refers to a means of communication such that the sensor may signal some portion of the article 20 when the sensor detects an input, preferably the means for initiating the vacuum for pulling urine into the receiving tubes 14. The sensor 12 may be separate from and operatively connected to another portion of the article 20, another sensor, an actuator, a controller or some other portion or component of the article 20. “Operatively connected” may, for example, include a means of communication such as an electrical connection via a conductive wire or member, via a transmitted signal such as radio frequency, infrared or another transmitted frequency communication. Alternatively, the sensor may be operatively connected via a mechanical connection such as a pneumatic or a hydraulic connection.

In article 20, for example, the sensor may be located in the front waist region 36, the rear waist region 38 or the crotch region 37 of article 20, and may be integral with, disposed adjacent to, joined to, or comprise a portion of the chassis 22, the topsheet 24, the backsheet 26, side panels 30, leg cuffs, a waist feature 34, a fastening system 40, etc. The sensor may be integral with the article 20, or may be installed by the caretaker or the wearer. The sensor may be completely contained within the article of the invention such as article 20 or may have a receiving portion located in the article such that it will come into contact with the desired input and another portion such as a transmitting portion located either in the article or outside the article. The sensor 20 may be external to the article 20 yet operatively connected to some portion of the article 20 such that the sensor may detect an input external to the article 20 and provide a signal to a controller and/or an actuator. In some embodiments, the sensor may be separate from the article, e.g., separately applied to some portion of the wearer, and/or may have one or more components separate from the article.

The sensor may further comprise a sensing “system” including two or more sensors, each of which may detect the same or different signals from the same or different sources. The sensing system may include components that are located inside, external to and/or separate from the article. For example, the sensing system may include a sensor inside the article that detects moisture and a sensor external to the article that detects motion, tension or muscle activity in the abdomen of the wearer. The sensing system may also or alternatively include components other than the sensing elements inside, external to and/or separate from the article. The sensing system, for example, may include a transmitter that is external to the article and transmits a signal to another part of the sensing system that is joined to or disposed in the article 20.

The article 20 preferably also comprises an actuator, which may be mechanical or electronic. As used in this application, the term “actuator” refers to a device that comprises “potential” and a means of transforming that potential to perform or activate a “responsive function.” The potential of the actuator may comprise either stored or potential energy or stored material. The actuator thus may perform or activate a responsive function by transforming potential energy to kinetic energy or by delivering a stored material. A “responsive function” is defined for the purposes of this application as a function performed upon the urine, the wearer, the article, or a component thereof. A device that merely provides a signal indicating that an event has occurred, however, is not considered an “actuator” as defined for the purposes of this application. A component of bodily waste, i.e., urine, may include, for example, moisture, electrolytes, enzymes, volatile gases, bacteria, etc. A component of the wearer may also include skin, genitalia, etc. A component of the article may also include leg cuffs, waist cuffs or other waste barriers and/or containment components, a fastening system, etc. Potential energy may be stored as mechanical, electrical, chemical or thermal energy. “Kinetic energy” as used in this application refers to the capacity to do work or to perform a responsive function as described above (e.g., initiating vacuum and pulling urine into receiving tubes and/or moving urine in receiving tubes into a collecting tube). “Delivery” for the purposes of the present invention refers to a mechanical delivery means such as expanding or moving elements, hydraulic effects, gas pressure differentials, valves, etc.

Triggering the creation of a three dimensional structure to capture waste, for example, involves responsive functions performed on a component of the article and, ultimately, on the waste. Capturing urine is a responsive function performed on the urine. Adjusting the article's geometry (in one, two or three dimensions) or physical properties (e.g., bending modulus, geometry, etc.) are examples of responsive functions, which may be performed on the article. Signaling a caretaker and/or the wearer that an event has occurred, however, does not perform a responsive function because it does not perform a function upon the waste—i.e., the urine, the wearer, the article or a component thereof. Signaling devices require an agent external to the system (e.g., a human, etc.) to act as an actuator to result in a function being performed. An actuator of an article of the invention may, for example, deliver a pump, air, heat, or store bodily waste; or trigger the release or creation of a structure or element designed to perform one or more of these functions or any other responsive function upon the waste, wearer, article, or a component thereof.

An actuator of the present invention may release potential energy to perform or activate a responsive function upon the urine, the wearer, the article, or a component thereof. The release of potential energy may transform mechanical, electrical, chemical and/or thermal potential energy into mechanical energy to perform the responsive function. In some preferred embodiments, electrical, chemical or thermal energy may assist or power a mechanical activator. Actuators may be triggered by a threshold level of an input to release potential energy to perform a responsive function or may respond continuously to an input as described below. An actuator of an article of the invention, for example, may include one or more of the following: pressurized gas, a pump, a closed system liquid transport member, etc. Potential energy may be stored in any manner sufficient to maintain/restrain it until it is required. Examples include batteries and/or capacitors, elastically, torsionally, compressively tensioned materials or structures, in the form of materials capable of performing physical functions (e.g., compressed gases, etc.).

Alternatively, an actuator of the present invention may comprise a quantity of a stored material that has the capacity to perform or activate a responsive function upon the urine, the wearer, the article, or any component or components thereof.

In one embodiment, for example, the actuator may actively deliver a stored material (such as air) that performs a responsive function (causes vacuum or suction). In this embodiment, the actuator may be triggered by a threshold level of an input to discontinuously deliver the stored material at a given time. The actuator may, for example, include a released high pressure gas (air), etc.

In alternative embodiments the sensor and/or actuator may comprise a closed system liquid transport member. A “closed system liquid transport member” or “transport member” comprises a liquid filled member having an inlet port and outlet port, which upon receipt of even a little amount of liquid at the inlet port practically immediately releases liquid at the outlet port. The liquid released from the outlet port may serve as an input signal to a sensor. For example, the liquid may be urine, which is released when the transport member—a receiving tube 14—imbibes urine at an inlet port or end. Liquid transport through such transport members is based upon direct suction rather than on capillarity.

There are preferably at least two regions within the transport member with different tube sizes, namely the one or more port region(s) having smaller tubes and the inner region having a much larger tube size.

The actuator may alternatively comprise an electrically sensitive gel. Electrically sensitive gels are polymeric gel networks that, when at least partially swollen with water, change volume and/or geometry (i.e., perform a mechanical function) under the application of an electric current or field. For example, certain partially ionized polyacrylamide gels will undergo anisotropic contraction of about 50% under weak electric fields (e.g., 0.5 volts/cm) when immersed in acetone and water. Alternative electrically sensitive gels may undergo electrically induced bending in the presence of water and a surfactant or may undergo an oscillating wave motion when subjected to an oscillating electric field. It is believed that local shrinkage may be induced in a portion of the gel, e.g., one side of a gel element, by concentrating positively charged surfactant molecules on the negatively charged gel polymer in an electric field. Changing the intensity and/or the polarity of the field induces a movement in the gel as one side decreases in length (e.g., a gel formed in a strip may curl). Electrically sensitive gels may comprise variable geometries such as rectangular, circular, reticulated grid, etc. patterns in order to provide a valve to release a material, allow a bodily waste to flow through, prevent a bodily waste from flowing through, encapsulate a bodily waste, etc. as they change volume and/or geometry. An exemplary material is a weakly cross-linked PAMPs gel (poly(acrylamido-2-methyl propane) sulphonic acid). This type of gel may perform various functions such as functioning as a valve to release a material. Other exemplary electrically sensitive gels are described in U.S. Pat. No. 5,100,933 issued to Tanaka on Mar. 31, 1990 and WO 9202005. Alternatively, pH sensitive gels or salt concentration sensitive gels that change volume and/or geometry at specific pH or salt concentrations, respectively, may be used as an actuator of the present invention.

An embodiment of an article of the present invention may include one or more proactive sensors and one or more actuators. By detecting an input signal prior to the impending event, a responsive system in the article may be triggered to prepare for the impending event. This will allow the construction of articles in which the waste-management technology is initially “hidden” or unobtrusive, but which is available at, or just before, the moment of need. Regardless of the specific input, the proactive sensor in these embodiments may trigger an actuator to perform an action on the bodily waste, the wearer, the article, or a component or components thereof to prepare for the occurrence of the event. For example, if an impending urination is to be detected via the electrical activity of the external muscles, the system is preferably triggered (i.e., the responsive system is activated) by a signal related to relaxation of the muscle. The actuator may then perform a function such as opening a valve to allow urine to flow into a storage device.

The actuator may be disposed in and/or operatively connected to any portion of the article of the invention that will allow the actuator to perform a responsive function upon the bodily waste, the wearer, the article, or a component thereof. In article 20, for example, the actuator may be located in the front waist region 36, the rear waist region 38 or the crotch region 37 of article 20, and may be integral with, disposed adjacent to or joined to a component of the chassis 22, the topsheet 24, the backsheet 26, side panels 30, leg cuffs, a waist feature 34, a fastening system 40, etc. The actuator 70 may also be completely contained within the article such as article 20, may have a portion located in the article and a portion located outside the article 20, or may be completely external to the article 20. An actuator or a portion of an actuator may be operatively connected to one or more sensors, one or more controllers, another portion of the actuator or another portion of the article 20. Further, the actuator may be integral with the article 20, or may be installed by the caretaker or the wearer.

The article 20 may also include a controller. A “controller” is defined for the purposes of this application as a device that receives an input from a sensor and determines if one or more actions are to be taken. The controller may receive a signal from the sensor and direct the actuator to perform a responsive function upon the bodily waste, the wearer, the article or a component thereof. Alternatively, the actuator may receive the signal directly from the sensor and perform a responsive function upon the wearer, the waste, the article or a component thereof A controller may include materials that undergo chemical or physical change, may be a chemical, mechanical or electrical device that processes information from a sensor, etc. An electrical controller that receives signals from the sensor such as electrical activity of muscles of the wearer may receive and monitor multiple electrical signals and may repeatedly trigger the actuator. The controller may be integral with the sensor component, integral with the actuator component, or a separate component of the system.

The controller may be disposed in and/or operatively connected to any portion of a disposable article that will allow the controller to receive a signal from the sensor and to provide a signal to the actuator. In article 20, for example, the controller may be located in the front waist region 36, the rear waist region 38 or the crotch region 37 of article 20, and may be integral with, disposed adjacent to or joined to the chassis 22, or a component of the topsheet 24, the backsheet 26, side panels 30, leg cuffs, a waist feature 34, a fastening system 40, etc. The controller may be integral with the article 20, or may be installed by the caretaker or the wearer. The controller 80 may be completely contained within the article such as article 20, may have a portion located in the article and a portion located outside the article, or may be located completely outside the article 20. A controller or a portion of a controller may be operatively connected to one or more sensors, one or more actuators, another portion of the controller or another portion of the article 20. The controller, for example, may receive a signal from the sensor and provide a signal to the actuator, e.g., by a radio frequency (rf) transmission.

Although distinct structural elements may perform the sensor, actuator, and controller functions, the sensor, actuator, and/or controller functions of the present invention need not be performed by distinct structural elements.

The article 20 of the present invention includes an actuator. The article 20 may include a discontinuous responsive system with or without a feedback control loop. The article 20 may alternatively include a continuous responsive system having a feedback control loop. A “responsive system” is defined for the purposes of this application as a system that includes a sensor and an actuator that acts upon the bodily waste, the wearer, the article, or a component thereof when the sensor detects the appropriate triggering input. Upon sensing a given input parameter, the actuator delivers a stored energy or material to perform a responsive function, i.e., acting upon the bodily waste, the wearer, the article, or a component thereof.

The responsive system of the present invention may respond in either a “continuous” or a “discontinuous” manner. As used in this application, a “continuous responsive system” refers to a responsive system in which the output is quantitatively dependent upon the quantity of the input, i.e., continuously increasing quantities of the input are required to effect continuously increasing quantities of the output, or where the output of the responsive system comprises a passive release of a stored material. For example, the tubes 14 provide a continuous response in which the output is quantitatively dependent upon the quantity of the input, i.e., as increasing quantities of urine contact the tubes 14, an increasing amount of the tubes 14 and 16 receive the urine until the capacity of the tubes or the container or drain receiving urine from the tubes is exhausted.

A “discontinuous responsive system,” however, refers to a responsive system that has an output function that is essentially independent of the quantity of the input beyond a threshold level. For example, when one or more threshold levels of a given input are met, the responsive system may release all or a pre-designated portion of its stored energy to perform a specific responsive function.

A responsive system of the present invention may include a single threshold level at which the responsive system may release all of its stored energy to perform a specific responsive function or may include multiple threshold levels at which the system may release a pre-designated portion of its stored energy to perform one or more specific responsive functions at each of the threshold levels. In an embodiment having a single threshold level, for example, the responsive system may release all of its stored energy to perform the entire responsive function when that threshold level is met. In such a single threshold embodiment, the discontinuous responsive system includes a system that has two states such as on or off. When a threshold quantity of an input such as bodily waste is present in the article, the responsive system may perform a single responsive function upon the waste, the wearer, the article or a component thereof, such as suctioning the waste away from the skin of the user. Thus, the discontinuous responsive system may perform a one-time “switch-like” function that changes from one state to another in the presence of a threshold level of an input.

The responsive system may also comprise a “closed loop” or an “open loop” system. A “closed loop” system, which is also referred to as a “feedback control loop” system, includes distinct sensor and actuator components and uses a detection or a measurement of an element or a parameter of the output condition as at least one trigger of a responsive function that is performed upon the input. The output condition may be the state of the input condition after the actuator has had the opportunity to perform a responsive function on the input condition. For example, if the sensor is monitoring pH in the article 20 and urine is discharged into the article 20 changing the pH of the system, i.e., the output condition of the responsive system, the responsive system may deliver a predetermined quantity of a pH buffer to bring the pH of the system back to the desired target pH or pH range or may release a buffer until the pH returns to the target pH or the pH range. The responsive function may be performed when the output condition reaches a threshold level, or may be performed only when the output condition and one or more other conditions are met. Acting upon the input may include acting upon the element sensed, e.g., sensing pH and acting upon the pH, or may include acting upon a composition of which the element sensed is an integral component, e.g., sensing an enzyme or moisture and acting upon it. As described above, a feedback control loop system includes at least two distinct components: the sensor and the actuator. The sensor detects an event, or a parameter associated with that event. The actuator receives a signal and performs a responsive function on the input condition detected by the sensor. The feedback control loop may further include a controller. In this case, the sensor provides a signal to the controller, and the controller directs the actuator to perform a responsive function upon the input condition. The controller may be a separate component of the responsive system or the controller function may be performed by the sensor and/or the actuator.

The feedback control loop may be “non-modulating” or “modulating.” In a “non-modulating” feedback control loop responsive system the responsive system acts as a one-time switch in which the actuator performs a responsive function on the input when the threshold level of the output condition is met. For example, the sensor may detect moisture from urine, and the actuator may release warm air and suction to capture the urine. The sensor may also detect a volatile gas that produces an offensive odor, and the actuator may release a release warm air and suction to capture the urine. In each of these examples, the actuator acts upon the input detected by the sensor. If the sensor detects urine and the actuator triggers warming of air, however, the actuator acts upon something other than the input detected by the sensor, i.e., acts upon the air instead of the urine and is therefore not a feedback control loop.

A “modulating” feedback control loop includes a sensor, an actuator and a controller. In a modulating feedback control loop, the output condition is monitored constantly or repeatedly, and the controller directs the actuator to perform a responsive function on the input in order to maintain the output condition at a desired set point or within a desired range. A modulating responsive system may constantly or repeatedly measure moisture from urine and deliver suction each time the moisture is detected above a threshold moisture level to provide a feedback control loop responsive system.

While particular non-limiting embodiments and examples of the present invention have been illustrated and described, it would be obvious to those skilled in the art that various other changes and modifications can be made without departing from the spirit and scope of the invention. For example, although the present invention is illustrated and described primarily with respect to a diaper type configuration, the present invention is not limited to this embodiment. The present invention may also be used, for example, in articles that are applied directly to a wearer prior to the application of the article of the invention or in place of a disposable diaper, in a pull-on diaper, a diaper insert, a sanitary napkin, a tampon, etc. It is therefore intended to cover in the appended claims all such changes and modifications that are within the scope of this invention.

I claim:

1. An apparatus for eliminating urine, comprising:
a plurality of receiving tubes for receiving the urine;
a central collecting tube associated with the receiving tubes for receiving the urine;
a vacuum source for suctioning the urine into the receiving tubes; and
a sensor for detecting the urine and for activating the vacuum source.

2. The apparatus of claim 1 further air associated with the vacuum source and a heater for heating that air.

3. The apparatus of claim 2 wherein the sensor is a moisture sensor.

4. The apparatus of claim 1 further comprising a container for collecting urine from the collecting tube.

5. The apparatus of claim 1 further comprising a drain for disposing of the urine collected in the collecting tube.

6. A method for keeping an incontinent or bedridden patient free of wetness from excretion of bodily waste fluids, comprising:
providing receiving tubes adjacent a source of moisture or wetness;
providing a central collecting tube associated with said receiving tubes for receiving moisture;
providing a vacuum source for suctioning moisture into said receiving tubes; and
providing a sensor for activating the vacuum source in the presence of moisture.

7. The method of claim 6 further comprising providing a heat source for warming air associated with the vacuum source.

8. The method of claim 6 further comprising providing a drain or container for collecting and/or disposing of bodily fluids in the collecting tube.
